(19) United States
(12) Patent Application Publication
(10) Pub. No.: US 2024/0132846 A1
(43) Pub. Date: Apr. 25, 2024

(54) METHOD FOR FORMING BIOCOMPATIBLE OSTEOBLAST AND CHONDROBLAST STEM CELL SHEETS FROM PD-L1 POSITIVE MESENCHYMAL STEM CELLS HAVING LOW IMMUNOGENICITY

(71) Applicants: NGOC BICH VU, HO CHI MINH (VN); PHUC VAN PHAM, HO CHI MINH (VN)

(72) Inventors: NGOC BICH VU, HO CHI MINH (VN); PHUC VAN PHAM, HO CHI MINH (VN)

(73) Assignees: VietNam National University Ho Chi Minh City, HO CHI MINH (VN); Stem Cell Institute, HO CHI MINH (VN)

(21) Appl. No.: 18/169,890

(22) Filed: Feb. 16, 2023

(30) Foreign Application Priority Data
Oct. 12, 2022 (VN) 1-2022-06603

(51) Int. Cl. C12N 5/077

(52) U.S. Cl. CPC C12N 5/0656; C12N 5/0654; C12N 2501/11; C12N 2501/113; C12N 2501/51; C12N 2501/599; C12N 2506/1392; C12N 2509/10; C12N 2513/00

(57) ABSTRACT

A method of manufacturing a stem cell sheets is provided which includes: (a) obtaining mesenchymal stem cells; (b) extracting programmed death ligands one (PD-L1) from the mesenchymal stem cells; (c) selecting only PD-L1 positive (PD-L1⁺ MSCs) from the PD-L1; (d) differentiating the PD-L1+ cells into osteoblasts and chondroblasts in a predetermined activation condition; and (e) forming the stem cell sheets by mixing the PD-L1+ MSCs with platelet rich plasma solution and CaCl₂).

### PRIORITY CLAIMS

This application claims priority under 35 U.S.C. § 119(a) of applications No. 1-2022-06603, entitled “T\hat{á}m t\hat{é} bào [symbol] [symbol] [symbol] có tính sinh mi\hat{\tilde{e}}n [symbol] ch th\hat{á}p”, filed on Dec. 10, 2022 in the Republic Socialist of Vietnam. The patent applications identified above are incorporated here by reference in their entirety to provide continuity of disclosure.

### FIELD OF THE INVENTION

The present invention relates generally to stem cell engineering. More specifically, the present invention relates to method of manufacturing biocompatible stem cell sheets that express low immunogenicity and high immunomodulation suitable for allogenic and autologous transplantations.

### BACKGROUND ART

Cartilage and bone damages are the most common injuries that cause severe effects on a patient's quality of life. Over the year, several approaches have been studied and developed to treat these conditions. Recently, mesenchymal stem cell (MSC) transplantation has emerged as a plausible approach to treat these injuries with promising results. There are various kinds of MSCs (in the forms of non-expanded or expanded cells) that can be used to treat cartilage damage. Allogenic osteoblasts and chondroblasts differentiated from mesenchymal stem cells are essential sources for cartilage and bone engineering. However, the predominant challenge of using osteoblasts and chondroblasts in the clinic is the immune rejection triggered in the host when these cells are transplanted to treat disease conditions, such as osteoarthritis and bone defects.

Currently, autologous MSCs from bone marrow and adipose tissue, as well as from peripheral blood, have been investigated to treat articular cartilage injuries. Wakitani et al. reported that transplantation of MSCs derived from bone marrow could improve osteoarthritis (OA).¹ Orozco et al. (2013) also used bone marrow-derived MSCs to treat 12 patients with OA. Their results showed that in 11 of the 12 patients, there were significant improvements in pain, disability, and quality of life.² Another clinical trial also used expanded MSCs from bone marrow to treat 15 patients with OA, and also showed that the treatment was safe and alleviated the patients' pain level and quality of life up to 4 years.³ Indeed, more than ten clinical trials from 2017 to date have shown improvement in pain and quality of life in OA patients treated with autologous bone marrow-derived MSCs.⁴ Moreover, autologous cells from adipose tissue also induced similar improvements in OA patients.⁵⁻¹⁰ Vega et al. (2015) performed a randomized controlled trial (RCT) to evaluate allogeneic stem cell treatment in 30 patients with OA. In this prio-art approach, the authors used the allogenic MSCs from bone marrow and compared them to control (hyaluronic acid injection). The results showed that there was a significant improvement in the stem cell treatment group compared to control group, without any side effects recorded.¹¹ In a recent meta-analysis review, Kim et al. (2020) analyzed six RCT studies consisting of 203 OA patients who received expanded MSCs as treatment.¹² The authors reported that intra-articular injection of the expanded MSCs without adjuvant surgery could improve pain for the OA patients, with a short-term follow-up (6-12 months). However, some evidence related to function and cartilage repair remain limited and unclear.¹² In an earlier systemic review of 17 studies, Ha et al. (2019) showed that intra-articular injection of MSCs also improved pain and function in the knee of OA patients.¹³ In addition to cartilage regeneration, MSCs have been used to treat bone defects or bone fractures in humans. 14-17 Gjerde et al. (2018) treated 11 patients with mandibular ridge resorption by using autologous expanded bone marrow MSCs. The results showed that bone marrow MSCs, in combination with biphasic calcium phosphate granules, significantly induced bone formation without side effects.¹⁷ In a recent report, Panicker et al. (2020) successfully used bone marrow stem cells with autogenous bone grafts to restructure craniofacial bone defects.¹⁸

However, clinical trials have shown that the treatment efficacy is poorer for late-stage OA (stage IV) compared to earlier stage (stage I-III).¹⁹ Indeed, in late-stage OA, the full thickness of cartilage is a factor and affects the ability of the MSCs to heal the lesions. Therefore, grafts of cartilage or bone tissues can be used in these cases. However, the resources of cartilage and bone tissues are limited from autologous sources as well as allogeneic donors. The use of osteoblasts and chondroblasts differentiated from MSCs can be a promising strategy to produce engineered cartilage and bone for clinical applications. The immune rejection after transplantation of allogenic osteoblasts and chondroblasts remains the biggest issue in the clinical usage of engineered cartilage and bone tissues from allogeneic MSCs.

Therefore, what is needed is a new method of extracting stem cells that have low immunogenicity.

What is needed is new stem cell materials or sheets consisting of PD-CL1⁺ that are suitable in allogenic and autologous transplantations.

Furthermore, what is needed is new materials and methods of isolating mesenchymal stem cells (MSCs) derived from human umbilical cord tissues by a novel explant procedure.

Finally, what is needed is a method of isolating PD-L1⁺ that is efficient and cost-effective.

The materials and the method disclosed in the present invention solve the above-described problems and objectives.

### SUMMARY OF THE INVENTION

Accordingly, an object of the present invention is to provide a method of manufacturing a stem cell sheets that includes: (a) obtaining mesenchymal stem cells; (b) extracting programmed death ligands one (PD-L1) from the mesenchymal stem cells; (c) selecting only PD-L1 positive (PD-L1⁺ MSCs) from the PD-L1; (d) differentiating the PD-L1⁺ cells into osteoblasts and chondroblasts in a predetermined activation condition; and (e) forming the stem cell sheets by mixing the PD-L1⁺ MSCs with platelet rich plasma solution and CaCl₂).

Another object of the present invention is to provide new stem cell materials or sheets consisting of PD-L1⁺ that are suitable in allogenic and autologous transplantations.

Another object of the present invention is to provide osteoblasts and chondroblasts that are different from PDL1 MSCs (PDL1-MSCs).

Another object of the present invention is to provide new materials and methods of isolating mesenchymal stem cells (MSCs) derived from human umbilical cord tissues by a novel explant procedure.

Another object of the present invention is to provide a method of isolating PD-L1⁺ that is efficient and cost-effective.

These and other advantages of the present invention will no doubt become obvious to those of ordinary skill in the art after having read the following detailed description of the preferred embodiments, which are illustrated in the various drawing figures.

### BRIEF DESCRIPTION OF THE DRAWINGS

The accompanying drawings, which are incorporated in and form a part of this specification, illustrate embodiments of the invention and, together with the description, serve to explain the principles of the invention.

FIG. 1 shows a method of forming a PD-L1⁺ sheet having a low immunology characteristics in accordance with an exemplary embodiment of the present invention;

FIG. 2A shows an osteoblast and chodroblast sheet obtained from the PD-L1⁺ mesenchymal stem cells (MSC) that completely proliferates to occupy the container in accordance with an exemplary embodiment of the present invention;

FIG. 2B shows an osteoblast and chodroblast sheet obtained from the PD-L1⁺ mesenchymal stem cells (MSC) that takes the shape of its container in accordance with an exemplary embodiment of the present invention;

FIG. 2C shows the resilient phenotype osteoblast and chodroblast sheet obtained from the PD-L1⁺ mesenchymal stem cells (MSC) in accordance with an aspect of the present invention;

FIG. 3A shows a scatter graph of the umbilical cord expressed PD-L1⁺ analyzed by flow cytometery after FACS sorting and fifth passage in accordance with an aspect of the present invention;

FIG. 3B shows a scatter graph of the umbilical cord expressed PD-L1⁺ analyzed by flow cytometery after FACS sorting and fifth passage in accordance with an aspect of the present invention;

FIG. 3C shows a count graph of the umbilical cord expressed PD-L1⁺ analyzed by flow cytometery after FACS sorting and more than fifth passages in accordance with an aspect of the present invention;

FIG. 3D shows a count graph of the umbilical cord expressed PD-L1⁺ analyzed by flow cytometery after FACS sorting and more than 5^{th} passages in accordance with an aspect of the present invention in accordance with an aspect of the present invention;

FIG. 4A-FIG. 4H show count graphs of the umbilical cord expressed PD-L1⁺ after differentiated into adipocytes versus different clusters of differentiation (CD) such as CD14, CD34, CD44, CD45, CD73, CD90, CD105, and Human Leukocyte Antigen D-related (HLA-DR) respectively in accordance with an aspect of the present invention in accordance with an aspect of the present invention;

FIG. 5A-FIG. 5G show the expression count graphs of PD-L1⁺ versus HLA-DR, CD40, CD80, CD86, PD-L1 MSC, PD-L1 Osteoblasts, and PD-L1 chondroblast respectively in accordance with an aspect of the present invention;

FIG. 6A-FIG. 6B show umbilical cord mesenchymal stem cells PLD-1⁺ (PDL1-MSCs) after differentiation into osteoblasts reacted with Alizarin Red dye and chondrocytes reacted with Safranin 0 dye, respectively in accordance with an aspect of the present invention;

FIG. 7A shows a gene expression level of osteoblast cell with different histochemical staining and real time PCR such as osteocalcin and osteopontin in accordance with an aspect of the present invention;

FIG. 7B shows a gene expression level of chondroblast cell with different histochemical staining and real time PCR such as aggrecan and collagen II in accordance with an aspect of the present invention; and

FIG. 8 shows a bar graph of cytokine concentrations for P <0.05 and P >0.05 for respective different groups such as control group, osteoblasts, chondroblasts, and PD-L1 MSC in accordance with an aspect of the present invention.

### DETAILED DESCRIPTION OF THE INVENTION

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well-known methods, procedures, and components have not been described in detail so as not to unnecessarily obscure aspects of the present invention.

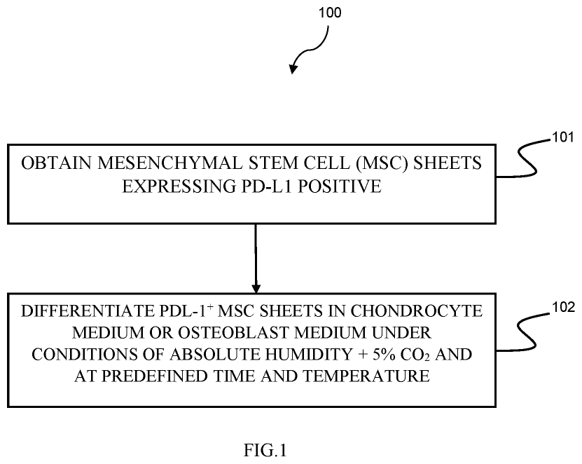

Now referring to FIG. 1, a method 100 for creating a low immunogenic chondrocyte and osteoblast sheets in accordance with exemplary aspect of the present invention is illustrated. In the present invention, mesenchymal stem cells (MSCs) which highly expressed programmed death-ligand 1 (PD-L1) are used for immunomodulation. Method 100 achieved the objectives of the present by obtaining mesenchymal stem cells (MSCs) that are positive with LD-L1 which can regulate the immune system of the host patients by silencing the immune activities of T-cells. Then LD-L1+ MSCs are formulated into sheets using platelet rich plasma and CaCl₂). These stem cell sheets with positive PD-L1 are differentiated into either osteoblasts or chondroblasts under predefined conditions, time, and temperature to achieve stem cell sheets. As a result, these stem cell sheets are biocompatible to transplantation without adverse side effects that hurt the host patients.

At step 101, mesenchymal stem cells (MSCs) sheets that express positive for PD-L1 against antibody are obtained. More particularly, first, mesenchymal stem cells (MSCs) suspension that express programmed death ligands (PD-L1) are first obtained (hereinafter as “PD-L1 MSCs”) are first obtained. In many aspects of the present invention, the PD-L1 MSCs are derived from umbilical cords, adipose tissues, bone marrows, blood vessels, placenta, and dental pulps. Then PD-L1 MSCs are further selected and sorted to contain more PD-L1. Thus, PD-L1 MSCs become PD-L1⁺ MSCs. Specifically, PD-L1⁺ MSCs are isolated from MSC derived PL-D1 cells using the flow cytometry techniques with different markers.

The PD-L1⁺ MSCs are applied to a platelet rich plasma solution in a container to suspend the PD-L1⁺ MSCs therein. The ratio between the number of cells and the volume of the platelet rich plasma solution is in the range of 0.1 million cells/ml to 10 million cells/ml. Then the platelet rich plasma solution and the PD-L1+ MSC are mixed together by pouring the cell suspension into a suitable mold and then add 5 μl to 20 μl of CaCl₂) solution. The CaCl₂) concentration is from 0.3 M to 1.8 M per ml of platelet rich plasma cell suspension. The solution is mixed well together. Next, the two solutions are gently and evenly shaken to avoid foaming. The mixing time does not exceed 60 second. The cell plate thickness is being controlled by the volume of the cell suspension and the area of the PD-L1+ MSCs sheet. A sheet of PD-L1+ MSCs is formed with a thickness between 100 μm to 3 mm. The container containing the mixture of cells and platelet rich plasma solution is placed in the incubator for 1 minute to 30 minutes to create a sheet of PD-L1+ MSCs that takes the shape of the container.

Continuing with step 101, more particularly, the platelet rich plasma solution is used to form fibrin substrates. Using 30% CaCl₂) solution to immerse the PD-L1+ MSCs into fibrin substrates in a short time period. PD-L1+ MSCs sheet is formed in the molding device after a period of 1 to 30 minutes. This time period is greatly shorter than that of other methods: usually from a few days to a few weeks. The small amount of CaCl2) solution eliminates chemical residues in the PD-L1+ MSC sheet for the bone or cartilage stem cell plate differentiation in the next step (i.e., differentiation step, see step 102). In addition, the CaCl2) solution also helps to reduce costs in the manufacturing process of both bone stem cell sheets and cartilage stem cell sheets.

Continuing with step 101, according to one aspect of the present invention, to obtain more PD-L1, MSCs are purified and sorted by the following steps: First, the MSCs with anti PL-D1 antibody bound fluorescence or magnetically bead bound anti-PL-D1 antibody are obtained. Accordingly, MSCs are stained with fluorescence binding anti-PLD-1 monoclonal antibody if the cells were separated by fluorescence activation. In the case fluorescence binding cell cleavage, 100,000 cells to 10 million MSCs are stained with fluorescence binding antibodies at concentrations of 0.1 μg/100 μl to 1 μg/100 μl for a period from 5 minutes to 60 minutes or in accordance with the manufacturer instructions. Next, MSCs were washed with phosphate buffered saline (PBS) or physiological saline (0.9% sodium chloride) 1 to 3 times by centrifugation at 500 rpm to 2,000 rpm for 3 min to 10 min to remove excess antibodies. After centrifugation, the supernatants are removed. The cell clusters at the bottom of the centrifuge tube are retained. The cell clusters are suspended in a separation buffer at a density of 1,000 cells/ml to 1 million cells/ml. Cell sorting with PD-L1 protein expression on systems such as fluorescence activated cell separation (FACS) system. The possible FACS used is FACSMelody, FACSJazz, FACSAria, or FACS Fusion from BD Biosciences.

Continuing again with step 101, MSCs after cleavage are collected and reanalyzed to evaluate the expression of PD-L1 proteins. In many aspects of the present invention, only samples containing 70% or more PL-D1+ MSCs with respect to specific markers are used for next step. Please refer to Experiment section. Next, PD-L1+ proliferation culture. PD-L1+ MSCs are suspended in serum and animal protein free culture media to obtain a density of 106 million cells per ml. These PD-L1+ MSCs are incubated in culture equipments such as plates or flasks so that the initial culture density is from 1,000 cells/cm2 to 25,000 cells/cm2 in an environment with absolute humidity and 5% CO2 at 37° C. Within the scope of the present invention, absolute humidity is defined as grams of water vapor (moisture) per cubic meter of inside the container regardless of the container temperature. When the PD-L1+ MSCs covered the surface of the culture equipment reach the coverage area from 70% to 100%, the harvesting process is carried out. The culture medium is removed and then added to a Deattachment cell separation solution in an amount that covers the entire surface. It is then incubated at room temperature for 1 minute to 10 minutes. The culture device is gently shaken to remove the cells from the surface. A straight pipette is used to wash the culture device surface. As a result, the PD-L1+ MSCs are separated from the culture surface. Next, the cell suspension is poured into a 15 ml to 50 ml centrifuge tube. Then the cell suspension is centrifuged at 1,500 to 3,500 rpm for 3 minutes to 10 minutes to collect the cell clusters. MSCs after harvesting are washed with phosphate buffer twice before proceeding to create PD-L1+ MSC plates.

In some aspects of the present invention, serum and animal protein free media includes Dubecco's Modified Eagle Medium/Nutrient Mixture F-2 (DMEM/F12) basal medium are supplemented with 1 ng/ml to 100 ng/ml of solution of Fibroblast Growth Factor (FGF) from 1 ng/ml to 100 ng/ml of EGF solution (Epideral Growth Factor), from 1 ng/ml to 100 ng/ml of PDG solution Platelet derived growth factor (PDG) from 1% to 3% albumin solution, and from 10 mM to 200 mM pyruvate acid.

Next, at step 102, PDL-1+ MSCs sheets are differentiated into either chondroblast or osteoblast sheets. In this step, PD-L1+ MSCs sheets obtained from step 101 are allowed to differentiate into chondroblasts and osteoblasts under specific activation conditions of the present invention.

More particularly, in the implementation of step 102, the PD-L1+ MSCs sheets are first placed in a suitable culture device and supplemented with medium to differentiate into chondrocytes using chondrocyte differentiation medium. On the other hand, PD-L1+ MSCs sheets are differentiated into osteoblasts using osteoblastic differentiation medium. Then, the above culture devices are placed in an environment specified by absolute humidity 5% of CO2 at the tempter of 37° C. It is noted again that absolute humidity is defined within the scope of the present invention as the measure of water vapor or moisture in the culture cabinet regardless of the temperature. Absolute humidity is expressed as grams of moisture per cubic meter of air (g/m3). The culture media re continually replaced with new ones. After 3 days up to 4 days, chondrocytes and osteoblastic are harvested after a culture period of 7 to 60 days in differentiation media.

In some aspects of the present invention, the chondrocyte differentiation medium includes DMDM/12 (Dulbecco's Modified Eagle Medium/Nutrient Mixture F-12) which is a basal medium supplemented with 5% to 20% insulin, human transferrin and selenious acid (ITS), and Premix Tissue Culture Supplement from 1 M to 10 M dexamethasone, 1 M to 10 M ascorbate-2-phosphate, 1% to 5% sodium pyruvate, and from 1 ng/ml to 100 ng/ml of transforming growth factor beta 1 (TGF-61) solution. The osteoblast differentiation medium includes Dubecco's Modified Eagle Medium/Nutrient Mixture F-12 (DMEM/F-12) which is a basal medium supplemented with 1% to 20% Fetal Bovine Serum (FBS) solution from 20 nM to 200 nM dexamethasone from 1 mM to 100 mM 6-glycerol from 1 μM to 100 μM ascorbic acide from 5 mg/ml to 60 mg/ml L-leucine and from 30 ng/ml to 300 ng/ml L-lysine. In these specific activation conditions, PD-L1+ MSCs sheets are differentiate into chondrocytes and osteoblasts sheets that express low immunogenicity.

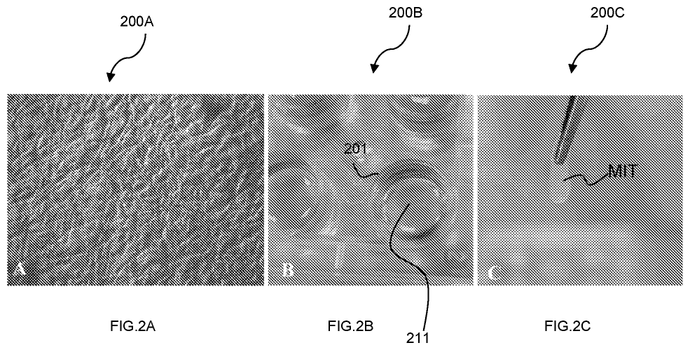

Referring now to FIG. 2A, a photograph 200A of osteocyte and chondrocyte plates generated from programmed death ligand 1 positive mesenchymal stem cells (PD-L1+ MSCs) plate after inoculation in accordance with an exemplary embodiment of the present invention is illustrated. As shown, photograph 200A is taken under a microscope which shows that PD-L1+ MSC plate spreads and proliferates on a substrate to form a sheet of PD-L1+ MSCs. Next, FIG. 2B represents a photograph 200B of a plate of osteoblasts 211 when cultured in a four well plate having four containers 201. It is conspicuous that the plate of osteoblasts 211 is plastic and take the shape of container 201. FIG. 2C shows phograph 200C of a plate of chondrocytes 212 after removed from container 201. It is shown that the phenotype of chondrocytes obtained from method 100 are resilient and elastic against gravity.

As shown in FIG. 2A-FIG. 2C and method 100, chondrocytes 211 is obtained from PD-L1+ antibody MSC differentiated in chondrocyte differentiation medium. Osteoblast 212 is obtained from PD-L1+ antibody MSC the osteoblast differentiation medium. They are both subject to conditions of absolute humidity 5% CO2, and at predetermined temperature and time. In many aspects of the present invention, MSCs are derived from umbilical cord, adipose tissue, bone marrow, cord blood, or dental pulp stem cell (DPSC). In addition, the MSCs are selected for PD-L1 using surface molecules as markers with high purity and population of 70% to 100%.

### EXPERIMENTS

Materials and methods Isolation of umbilical cord-derived mesenchymal stem cells MSCs derived from human umbilical cord tissues were isolated by explant culture, per previously published, with some modifications.23, 30, 31 The human umbilical cord tissues were taken from the donors with consent. Only the samples negative for HIV, HBV, and HCV were selected for stem cell isolation. The tissues were washed twice with Washing Buffer with 1× antibiotic-mycotic (Regenmedlab, Ho Chi Minh City, Vietnam). The tissues were then dissected into small (1-2 mm2) fragments, and placed into T-75 cm2 flasks. MSCCult I Primary medium (5 mL; Regenmedlab) was added to the flasks for five days. Then, 10 mL of fresh culture medium (MSCCult I Primary medium) was replenished every five days until the cell confluency reached 70-80%. MSCCult I Primary is a mesenchymal stem cell culture medium that is optimized for culture the primary cells isolated from adipose tissue, umbilical cord, bone marrow, etc. This product available in the market such as Biomedmart, Rengemadlab, etc. The cells were detached (using Deattachment reagent; Regenmedlab), and continuously subcultured or grown until the 5th passage. At which time they were collected for the cell sorting assay.

PDL1-positive cell were sorted by Fluorescent Activated Cell Sorter (FACS). These cells (PD-L1+ MSCs) were stained with anti-PDL1 monoclonal antibody conjugated with Phycoerythrin (PE) (Abcam, Cambridge, UK) for 20 min at room temperature. Anti-PDL1 (PE) is an logiomeric protectin complex from red algae that exhibits intensely bright red-orange fluorescent with high quantum yields. The stained cells were washed twice with phosphate buffered saline (PBS; Regenmedlab). The cells were analyzed and sorted in a cell sorter such as FACS Melody Cell Sorter; BD Biosciences, San Jose, CA, USA.

The cells (PD-L1+ MSCs and cells are used interchangeably) were kept at 37° C. during the sorting procedures. The sorted cells were kept in the after-sort buffer (i.e. culture medium supplemented with 2× antibiotic-mycotic, and prewarmed to 37° C. before use). The sorted cells were directly transferred into a T-75 flask for culture without any centrifugation. After 24 hours, the culture medium was replenished with fresh medium. The cells were subcultured until cell confluency reached 70%. At the time, these cultured sorted cells were collected for reconfirmation of PDL1 expression by flow cytometry.

Sorted cells were expanded for two more passages in order to obtain enough cells for the subsequent experiments. Please refer to step 101 above. Before use in these next experiments, they were characterized for the MSC phenotype, per guidelines suggested by the International Society of Cell and Gene Therapy (ISCT).32

The expression of CD14-FITC, CD34-FITC, CD44-PE, CD45-APC, CD73-PE, CD90-PE, CD105-PE, and HLA-DR-FITC on the surface of the sorted cells were assessed by flow cytometry. The cells were stained with monoclonal antibodies for each of the respective markers (BD Biosciences). Then, the cells were washed twice with FACS Wash Buffer to remove the unbound antibodies before they were analyzed on a flow cytometer (FACSCalibur, BD Biosciences). The data were analyzed by FlowJo software (BD Biosciences). The differentiation potential of the cells were confirmed by induction in osteogenesis inducing medium (for osteoblasts), adipogenesis-inducing medium (for adipocytes), and chondrogenesis-inducing medium (for chondroblasts). The three differentiation media were purchased from Thermo-Fisher (Waltham, MA, USA). The adipocyte phenotype was confirmed by Oil Red staining. Alizarin Red staining and Safranin O staining were used to confirm the phenotype of osteoblasts and chondroblasts, respectively. The stains were obtained from Sigma-Aldrich (St Louis, MO, USA).

Differentiation and characterization of osteoblasts and chondroblasts PD-L1-MSCs were induced into osteoblasts using the osteogenesis kit (Thermo-Fisher) and into chondroblasts using the chondrogenesis kit (Thermo-Fisher). The protocols were carried out according to the manufacturers' instructions. After 21 days of induction, the differentiated cells were assayed for the osteoblast phenotype (by Alizarin Red staining) and the chondroblast phenotype (by Safranin O staining), respectively. The osteoblast phenotype was also confirmed by the expression of osteocalcin and osteopontin by real-time RT-PCR. The chondroblast phenotype was also confirmed by the expression of aggrecan and collagen type II by real-time RT-PCR. The RT-PCR reactions were performed according to instructions of the commercial kits (RT SYBR Realtime One-step Kit; Invitrogen, Carlsbad, CA). The total RNA from PD-L1+ MSCs was used as a control for all the assays. Gene expression was normalized against the internal control (GAPDH). The differentiation efficacies of PD-L1+ MSCs into osteoblasts and chondroblasts were evaluated based on the expression of osteocalcin and collagen type II, respectively, and also based on flow cytometry. The differentiated cells were permeabilized and fixed, per instructions of the commercial kits (CytoFix/CytoPerm, BD Biosciences). Next, osteoblasts were stained using anti-osteocalcin PE (Abcam, Cambridge, MA), and chondroblasts were stained with anti-collagen type II-PE (Abcam) for 30 min at room temperature. The cells were washed twice before resuspension in sheath fluid for analysis by flow cytometer (FACSCalibur, BD Biosciences). The data was analyzed by FlowJo software (BD Biosciences).

The expression of PDL1, HLA-DR, CD40, CD80, and CD86 on the surface of PDL1-MSCs, osteoblasts, and chondroblasts was evaluated by flow cytometry. The cells were stained with anti-PDL1-PE, HLA-DR-FITC, CD40-FITC, CD80-FITC, and CD86-FITC monoclonal antibodies (BD Biosciences) in staining buffer for 20 min in the dark at room temperature. Then, the cells were washed twice with PBS to remove the unbound antibodies. The stained cells were resuspended in 200 μL of sheath fluid, and run and analyzed on a FACSCalibur flow cytometer. As above, the results were analyzed by FlowJo software. Mixed lymphocyte reaction (MLR) and lymphocyte proliferation assessment. The suppression of osteoblasts, chondroblasts, and PDL1-MSCs on allogenic lymphocytes was evaluated using mixed lymphocyte reactions (MLRs). All assays were performed according to the instructions of the commercial kits (Immunomodulation Potency MSC kit, Regenmed lab). Briefly, the osteoblasts, chondroblasts, and PDL1-MSCs were plated overnight in 96-well plates, at 103 cells per well, and then treated with mitomycin C to inhibit their proliferation. The peripheral blood mononuclear cells (PBMCs) were collected using the MNC collection tube (supplied in the kit) from 15 mL of peripheral blood. The PBMCs were stimulated with 2.5 μg/mL of phytohemag glutinin (PHA). Stimulated PBMCs were added into the 96-well plates with cells at 3 densities: 103, 104, and 105 cells per well. The wells with osteoblasts, chondroblasts, or PDL1-MSCs (but without PBMCs) were used to correct the signals by cells (negative controls). The wells with PBMCs (but without osteoblasts, chondroblasts, or PDL1-MSCs) were considered as positive controls. The plates were incubated for 24 hours before alamarBlue™ dye was added to measure the proliferation of PBMCs on a DTX880 biophotometer (Beckman Coulter, Brea, CA, USA). The OD values were normalized to negative controls before the values were used for comparison among the groups. All experiments were performed in triplicate. ELISA for quantification of TNF-α and IFN-γ The supernatants from the experimental groups were collected and centrifuged at 2000×g for 15 min to obtain the cell-free supernatants. These supernatants were kept frozen at −96° C. until use in enzyme-linked immunosorbent assay (ELISA) to measure cytokine production. The ELISAs for IFN-γ and TNF-α were conducted following the suppliers' instructions (Abcam). Statistical analysis. The data were analyzed for statistical significance using GraphPad Prism (GraphPad Software, Inc., La Jolla, CA, USA). Data were presented as mean and standard deviation. When applicable, a Student's unpaired t-test and oneway ANOVA were used to determine significance; P<0.05 was considered to be statistically significant.

Method 100 for creating low immunologic chondrocytes from human umbilical cord mesenchymal stem cells was performed. First, programmed death ligand 1 mesenchymal stem cells (PDL-1 MSC) in a human umbilical cord was isolated. Next, the MSCs are stained with PE-labeled anti PLD-1 antibody in the ratio of 106 MSCs per 100 ng (nano gram) of phycoerythrin (PE) labeled anti PLD1 antibody. The stained MSC was then incubated for 30 minutes at room temperature.

Afterwards, the MSCs were washed twice with phosphate buffer saline (PBS). The washed mixture was centrifuged at 300 rpm for 10 minutes to remove excess antibody and collect cell residues. Next the cell residues were suspended in a Deattachment cell separation buffer at a density of 106 cells/ml. PD-L1+ cells were then isolated on a fluorescence activated cell separation system such as FACSMelody manufactured by BD-BioScience Company based on the phycoerythrin (PE) fluorescence signal. Finally, MSCs positive for PD-L1 were obtained. This final product is also known as PD-L1+ MSC. The purity of the PD-L1+ samples were re-analyzed to achieve a purity of 70% or more which was necessary to conduct proliferation in a suitable culture medium.

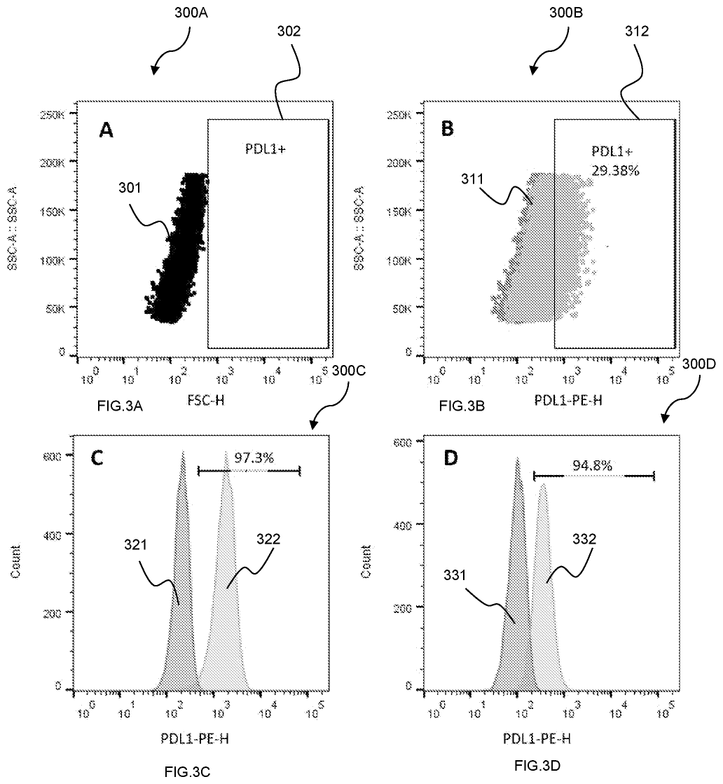

Next referring to FIG. 3A, a scatter graph 300A of umbilical cord MSCs that are positive for PD-L1, also known as PD-L1+ MSCs, were analyzed by flow cytometry in accordance with an exemplary embodiment of the present invention is illustrated. The flow cytometry of the samples is performed with the Fluorescent Activated Cell Sorters (FACS) on the FACSCalibur device. It is noted that it is well known from the flow cytometry analysis using FACS, the ordinate axis is the side scatter with area gated (SSC-A) and the abscissa axis is the forward scatter with height gated (FSC-H). After steps 101 to 102 above were performed, the PD-L1+ MSCs were collected. These PD-L1+ MSCs were subcultured for five times to obtain a sufficient amount of MSC for cell sorting procedure. After the FACSCalibur analysis, scatter graph 300A shows that umbilical cord MSC PDL-1+ MSCs 302 were discerned from an isotype control 301. Next, FIG. 300B shows a scatter graph 300B of the differentiation between PD-L1 MSCs and PL-L1+ MSCs 312. Graph 300B shows that only 29.38% of PD-L1+ MSC 312 was successfully sorted from a total solution 311 consisted of PD-L1 MSCs and PD-L1+ cells. Next, in FIG. 3C, a histogram representation 300C of the flow cytometry analysis of PD-L1+ MSCs 322 versus reference sample 321. The gated population of PD-L1 revealed that approximately 97.3% of PDL-1+ MSC 322 is reconfirmed. In FIG. 3D, a histogram presentation 300D of PD-L1+ MSCs 332 versus another reference sample 331. This time PD-L1+ MSCs 321 were subcultured for 2 more times to collect enough cells for the next flow cytometry analysis. These cells were reconfirmed for PD-L1+ expression at 94.8%. As seen, method 100 of the present invention shows positive expression for PD-L1 MSCs.

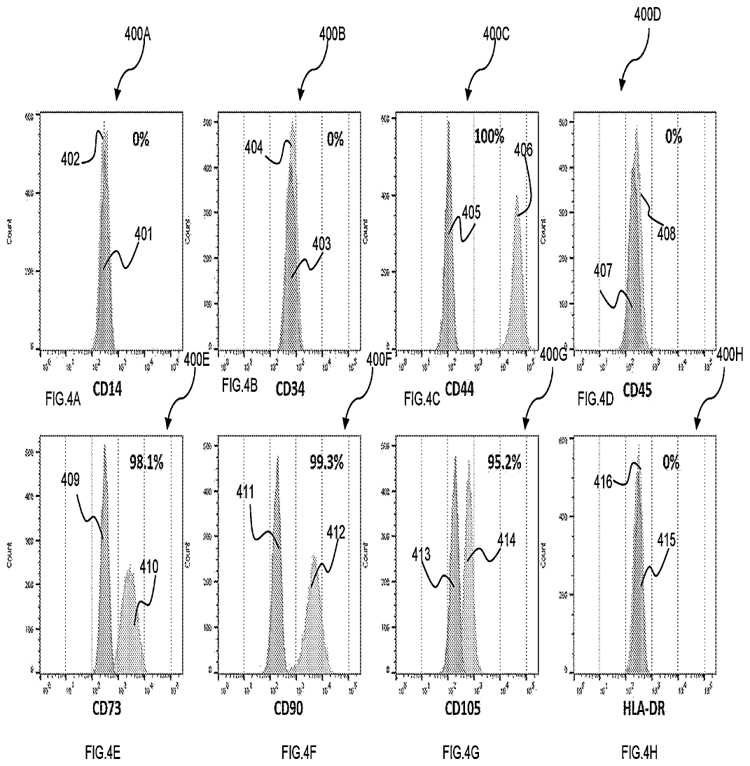

Next referring to to FIG. 4A-FIG. 4H, histogram representations 400A-400H of PD-L1+ MSCs characterized the phenotype of MSC that shows differential expressions (correlations) with different markers are illustrated. It is noted that PD-L1+ MSCs can be defined by expressions of a set of cellular surface markers. In FIG. 4A, histogram 400A shows negative expression of 0% between HD-L1+ 402 and CD14 401 (glycosylphophatidyllimsitol or GPI). In FIG. 4B, histogram 400B shows negative expression of 0% between HD-L1+ 404 and CD34 403 (transmembrane glycoprotein). In FIG. 4C, histogram 400C shows positive expression of 100% between HD-L1+ 406 and CD44 405 (antigen cell-surface glycoprotein). In FIG. 4D, histogram 400D shows negative e expression of 0% between HD-L1+ 408 and CD45 407 (lymphocyte common antigen). In FIG. 4E, histogram 400E shows positive expression of 98.1% between HD-L1+ 410 and CD73 409 (lymphocyte differentiation antigen). In FIG. 4F, histogram 400F shows a positive expression of 99.3% between HD-L1+ 412 and CD90 411 (GPI). In FIG. 4G, histogram 400G shows a positive expression between HD-L1+ 414 and CD105 413 (cell membrane glycoprotein). In FIG. 4H, histogram 400H shows negative expression of 0% between HD-L1+ 416 and human leukocyte antigen-DR (HLA-DR) 415. These results from flow cytometry showed that PD-L1+ MSCs were indeed umbilical cord stem cells that are positive for PD-L1.

Next, to form PD-L1+ MSCs sheets, umbilical cord PD-L1+ MSCs were suspended in a serum and animal protein free culture medium to obtain a density of 106 cells per ml. In various aspects of the present invention, this culture is consisted of: Dulbecco's Modified Easgle Medium/Nutrient Mixture F-12 (DMEM/F12). This basic medium was supplemented with 1 ng/ml to 100 ng/ml solution of Fibroblast Growth Factor (FGF) and 1 ng/ml to 100 ng/ml of EGF Epidermal Growth Factor (EGF). 1 ng/ml to 100 ng/ml solution of from of platelet derived growth factor) (PDG), 1% to 30% albumin solution, and 10 mM to 300 mM pyrivate acid. These cells are cultured in T-75 cell culture flasks (T-75 flasks) at the density of 5,000 cells/cm2 under the conditions of 37° C. and 5% CO2 and absolute humidity. When the cells had covered at least 70% surface of the T-75 flask, they were harvested. Then the culture medium was aspirated. Next 10 ml of Deattachment cell separation solution was added to each T-75 flask. The T-75 flasks were incubated at room temperature for 5 minutes. These T-75 flasks were gently shaken to remove the cells from the surface. Next step, the surface of the T-75 flask was flooded using a straight pipette to separate the cells from the culture surface. The cell suspension was collected into 50 ml centrifuge tubes. These tubes were centrifuged at 500 rpm for 10 minutes and the cell residues were collected. After the harvest, the cells were washed with phosphate buffer saline (PBS) twice.

Next referring again to FIG. 2B, the PDL-1+ MSCs sheets were generated by the following steps: First, 106 PD-L1+ MSCs explanted from umbilical cord were poured into a 1 ml of platelet rich plasma solution. Then 10 μl of 20% CaCl2 was added to the platelet rich plasma cell suspension. 1 ml of this cell suspension was pipetted into each well of the 6 well plates. The umbilical cord PD-L1+ MSCs were formed in the well in the shape of a well about 0.5 mm thick after 20 minutes. This is the implementation of step 101 of the presentation.

Finally, umbilical cord PD-L1+ MSCs are differentiated into chondrocyte plate. First place the umbilical cord MSC PD-L1+ plate in each well of the 6 well plate. Each well plate is supplemented with 3 ml of MSC chondrocytes differentiation medium. The culture plate is placed in the cell culture cabinet at 37° C. and 5% CO2 at absolute humidity. The new medium is continuously replaced every 3 days for 21 days. PD-L1+ MSC chondrocytes plate is harvested after 21 days of differentiation. This is the implementation of step 102 of the presentation.

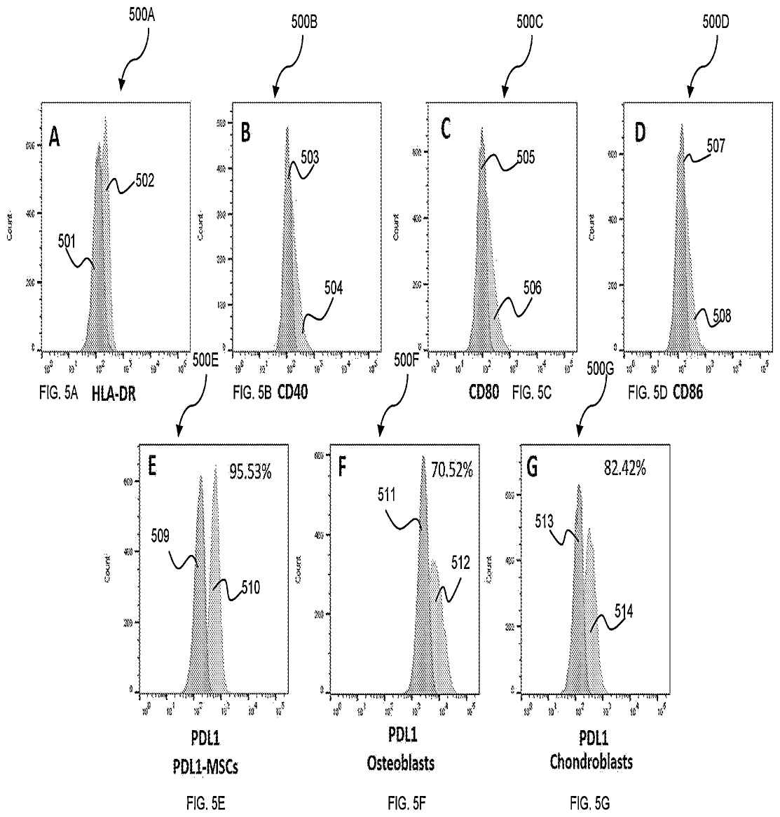

Next referring to FIG. 5A-FIG. 5G, histogram representations 500A-500G showing the correlations between umbilical cord derived PD-L1+ MSCs and PD-L1 osteoblasts, PD-L1 chondroblast, HLA-DR, CD40, CD80, and CD86. In FIG. 5A, a graph 500A indicates a slight differentiation between a HLA-DR 501 and PD-L1+ 502. In FIG. 5B, a graph 500B indicates a slight differentiation between CD40 (transmembrane protein) 503 and PD-L1+ 506. In FIG. 5C, a graph 500C indicates a slight differentiation between CD80 (membrane protein in the immunoglobulin) 505 and PD-L1+ 506. In FIG. 5D, a graph 500D indicates a slight differentiation between CD86 (costimulatory molecule in eliciting T-cell) 507 and PD-L1+ 508. Accordingly, there is a slight increase in HLA-DR, CD40, CD80 and CD86 in bone and chondrocytes compared with PD-L1+ umbilical cord with statistically significant P >0.05) In FIG. 5E, a graph 500E indicates a differentiation of 95.53% between programmed death ligand 1 from mesenchymal stem cells (PD-L1-MSCs) 509 and MSC PD-L1+ 510. In FIG. 5F, a graph 500F indicates a differentiation of 70.52% between PD-L1 osteoblasts (bones) 511 and PD-L1+ 512. In FIG. 5G, a graph 500G indicates a differentiation of 95.53% between PD-L1 chondroblasts 513 and PD-L1+ 514. As sheen, graphs 500E to 500G show significant reduction in PD-L1 expression in the differentiated osteocytes and chondrocytes when compared with PD-L1 MSCs with statistically significant P<0.05. However, expression of PD-L1 was noted in 70.52±7.31% in the differentiated osteocytes and 82.42±14.56% in differentiated chondrocytes when compared with 95.35±2.03% in PD-L1 MSCs.

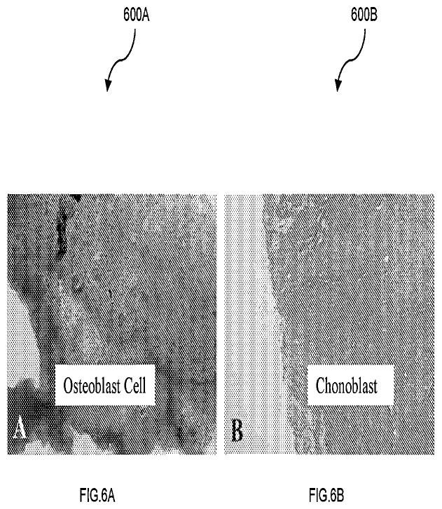

Next referring to FIG. 6A, a phograph 600A shows umbilical cord mesenchymal stem cells PLD-1+(PDL1-MSCs) after differentiation into osteoblasts when reacted with Alizarin Red. In FIG. 6B, a photograph 600B shows umbilical cord mesenchymal stem cells PLD-1+(PDL1-MSCs) after differentiation into chondrocytes when reacted with Safranin O dyes.

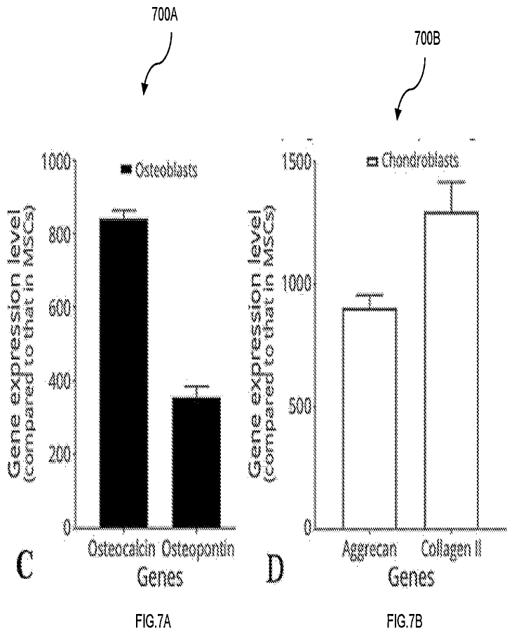

Referring now to FIG. 7A, a bar graph 700A of gene expression level between PD-L1 MSCs and osteocalcin genes as compared to osteopontin. Accordingly, osteocalcin specific genes accounts for from 842.41±21.21% and osteopontin 356.19±28.31% in osteoblast differentiated PD-L1 MSC. Similarly, referring to FIG. 7B, a bar graph 700B of gene expression level between PD-L1 MSCs and chondroblasts including aggrecan as compared to collagen II. As shown in bar graph 700B, aggrecan accounts for 356.19±28.31% and type II collagen accounted for 1294.89±121.43%. According to the results of flow cytometry analysis PD-L1 MSCs differentiated into osteoblasts were positive (78.41±8.56% in total number of cells) for osteocalcin, while PD-L1 MSC differentiated into chondrocytes were positive (82.89±8.41% of total cells) for type II collagen.

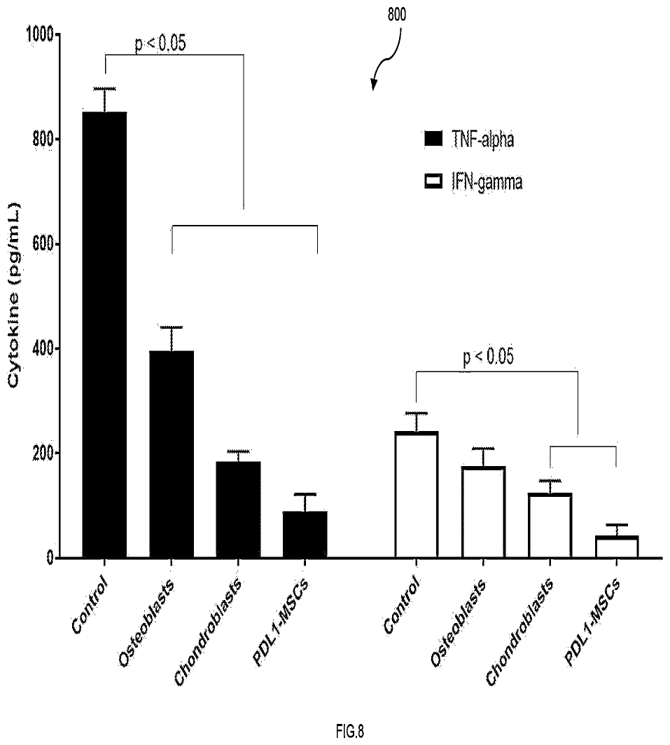

Referring next to FIG. 8, a bar graph 800 of cytokine concentration (pg/mL) with respect different groups and growth factors in accordance with exemplary embodiment of the present invention is illustrated. Graph 800 shows low immunogenicity of chondrocytes and bone cells differentiated from PD-L1+ umbilical cord mesenchymal stem cells. Pro-inflammatory factors including tumor necrosis factor alpha (TNF-α) levels were also significantly decreased in all media where lymphocytes were co-cultured with differentiated osteoblasts from PD-L1+ umbilical cord MSC, chondrocytes differentiated from umbilical cord MSC PD-L1+(PD-L1 MSCs). Specifically, the concentration of TNF-α was 852±44.49 μg/ml in the control (cell-free with PHA phytohemagglutinin), and was 395.59±45.21 μg/ml in differentiated osteocytes from cells., MSC PD-L1+, reaching 183.89±45.21 μg/ml in chondrocytes differentiated from PD-L1 cord MSC and reaching 89.31±32.57 μg/ml in umbilical cord MSC PD-L1+.

Continuing with FIG. 8, the levels of pro-inflammatory factor interferon gamma (IFN-γ) levels are also significantly reduced in all media where lymphocytes were co-cultured with differentiated osteoblasts from PD-L1+ umbilical cord MSC, chondrocytes differentiated from umbilical cord SMC PD-L1+, or umbilical cord MSC PD-L1+. Specially, IFN-γ levels were reduced from 241.41±35.21 μg/ml in the control (non cells with PHA phytohemagglutinin) to 175.45±34.11 μg/ml in differentiated osteocytes from umbilical cord mesenchymal stem cells PD-L1+ to 124.31±23.48 μg/ml in chondrocytes differentiated from umbilical cord MSC PD-L1+ and to 42.47±21.21 μg/ml in umbilical cord MSC PD-L1+. This shows that chondrocytes and osteocytes differentiated from umbilicord MSC PD-L1+ of the present invention have very low immunogenicity.

### CONCLUSION

The present invention provides for the manufacture of chondrocytes and osteoblasts from various types of MSC with low immunogenicity, for the treatment of cartilage and bone injuries in humans. The method and products of the present invention help to proactively avoid rejection after allograft transplantation of chondrocytes and osteoblasts on a large scale. Therefore, the cost of treatment will be greatly reduced the quality of the cell sheet is more strictly controlled. Patients need not have to wait a long time to create autologous chondrocytes and osteoblasts. At the same time, the product is made with elasticity and flexibility. Flexible implantation in different positions with the size and shape can be adjusted arbitrarily.

Regarding the time to create a cartilage or bone cell plate, the present invention uses platelet rich plasma to create fibrin matrix and put the cells in a fibrin substrate in a relatively short time from 15-60 minutes. So the sufficient numbers of cells adhere, proliferate and differentiate on a significantly shortened substrate as compared to other methods. In term of costs, method 100 of the present invention minimizes the costs at the stage of making fibrin substrates by using only CaCl₂) in a very small amount.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should, therefore, be construed in accordance with the appended claims and any equivalents thereof.

### Abbreviations

PD-L1 Programmed Death Ligand 1

PD-L1+ Programmed Death Ligand Positive

PBS phosphate buffer saline

PE Phycoerythrin

MSC Mesenchymal Stem Cells

CD Cluster of Differentiation

OA Osteoarithis

RCT Randomized Controlled Trial

FACS Fluorescent Activated Cell Sorters

HLA-DR Human Leukocyte Antigen D Related

PBMC Peripheral Mononuclear Cells

CaCl₂) Calcium Chloride

ELISA Enzyme Linked Immunosorbent Assay

IFN-γ Interferon gamma

TFN-α Tumor Necrosis alpha

DMEM/F12 Dulbecco's Modified Easge Medium/Nutrient Mixture F12

EGF Epidearal Growth Factor

PDG Platelet Derived Growth Factor

SSC Side Scatter

FSC Forward Scatter (90σ with the laser light)
GPI glycosylphosphatidylimsitol

### REFERENCES

1. Wakitani S, Imoto K, Yamamoto T, Saito M, Murata N, Yoneda M. Human autologous culture expanded bone marrow mesenchymal cell transplantation for repair of cartilage defects in osteoarthritic knees. Osteoarthritis Cartilage 2002; 10:199-206.
2. Orozco L, Munar A, Soler R, Alberca M, Soler F, Huguet M, et al. Treatment of knee osteoarthritis with autologous mesenchymal stem cells: a pilot study. Transplantation 2013; 95:1535-41.
3. Soler R, Orozco L, Munar A, Huguet M, Lopez R, Vives J, et al. Final results of a phase I-II trial using ex vivo expanded autologous Mesenchymal Stromal Cells for the treatment of osteoarthritis of the knee confirming safety and suggesting cartilage regeneration. Knee 2016; 23:647-54.
4. Al-Najar M, Khalil H, Al-Ajlouni J, Al-Antary E, Hamdan M, Rahmeh R, et al. Intra-articular injection of expanded autologous bone marrow mesenchymal cells in moderate and severe knee osteoarthritis is safe: a phase I/II study. J Orthop Surg Res 2017; 12:190.
5. Jo C H, Chai J W, Jeong E C, Oh S, Shin J S, Shim H, et al. Intra-articular Injection of Mesenchymal Stem Cells for the Treatment of Osteoarthritis of the Knee: A 2-Year Follow-up Study. Am J Sports Med 2017; 45:2774-83.
6. Jo C H, Lee Y G, Shin W H, Kim H, Chai J W, Jeong E C, et al. Intraarticular injection of mesenchymal stem cells for the treatment of osteoarthritis of the knee: a proof-of-concept clinical trial. Stem Cells 2014; 32:1254-66.
7. Lee W S, Kim H J, Kim K I, Kim G B, Jin W. Intra-Articular Injection of Autologous Adipose Tissue-Derived Mesenchymal Stem Cells for the Treatment of Knee Osteoarthritis: A Phase IIb, Randomized, Placebo Controlled Clinical Trial. Stem Cells Transl Med 2019; 8:504-11.
8. Hong Z, Chen J, Zhang S, Zhao C, Bi M, Chen X, et al. Intra-articular injection of autologous adipose-derived stromal vascular fractions for knee osteoarthritis: a double-blind randomized self-controlled trial. Int Orthop 2019; 43:1123-34.
9. Nguyen P D, Tran T D, Nguyen H T, Vu H T, Le P T, Phan N L, et al. Comparative Clinical Observation of Arthroscopic Microfracture in the Presence and Absence of a Stromal Vascular Fraction Injection for Osteoarthritis. Stem Cells Transl Med 2017; 6:187-95.
10. Bui K H, Duong T D, Nguyen N T, Nguyen T D, Le V T, Mai V T, et al. Symptomatic knee osteoarthritis treatment using autologous adipose derived stem cells and platelet-rich plasma: a clinical study. Biomed Res Ther 2014; 1:2-8.
11. Vega A, Martin-Ferrero M A, Del Canto F, Alberca M, Garcia V, Munar A, et al. Treatment of Knee Osteoarthritis With Allogeneic Bone Marrow Mesenchymal Stem Cells: A Randomized Controlled Trial. Transplantation 2015; 99:1681-90.
12. Kim S H, Djaja Y P, Park Y B, Park J G, Ko Y B, Ha C W. Intra-articular Injection of Culture-Expanded Mesenchymal Stem Cells Without Adjuvant Surgery in Knee Osteoarthritis: A Systematic Review and Meta-analysis. Am J Sports Med 2020; 48:2839-49.
13. Ha C W, Park Y B, Kim S H, Lee H J. Intra-articular Mesenchymal Stem Cells in Osteoarthritis of the Knee: A Systematic Review of Clinical Outcomes and Evidence of Cartilage Repair. Arthroscopy 2019; 35:277-288.e2.
14. Dilogo I H, Kamal A F, Gunawan B, Rawung R V. Autologous mesenchymal stem cell (MSCs) transplantation for critical-sized bone defect following a wide excision of osteofibrous dysplasia. Int J Surg Case Rep 2015; 17:106-11.
15. Thua T H, Bui D P, Nguyen D T, Pham D N, Le Q B, Nguyen P H, et al. Autologous bone marrow stem cells combined with allograft cancellous bone in treatment of nonunion. Biomed Res Ther 2015; 2:1-9.
16. Fernandez-Bances I, Perez-Basterrechea M, Perez-Lopez S, Nunez Batalla D, Fernandez Rodriguez M A, Alvarez-Viejo M, et al. Repair of long-bone pseudoarthrosis with autologous bone marrow mononuclear cells combined with allogenic bone graft. Cytotherapy 2013; 15:571-7.
17. Gjerde C, Mustafa K, Hellem S, Rojewski M, Gjengedal H, Yassin M A, et al. Cell therapy induced regeneration of severely atrophied mandibular bone in a clinical trial. Stem Cell Res Ther 2018; 9:213.
18. Panicker P P, Mohan S P, Nallusamy J, Lakshmi S J, Johny J, Bhaskaran M K. Reconstruction of Craniofacial Bone Defects with Autologous Human Bone Marrow Stem Cells and Autogenous Bone Grafts: A Case Report with Review of Literature. J Pharm Bioallied Sci 2020; 12(Suppl 1):5394-8.
19. Kim J D, Lee G W, Jung G H, Kim C K, Kim T, Park J H, et al. Clinical outcome of autologous bone marrow aspirates concentrate (BMAC) injection in degenerative arthritis of the knee. Eur J Orthop Surg Traumatol 2014; 24:1505-11.
20. Wang P, Li Y, Huang L, Yang J, Yang R, Deng W, et al. Effects and safety of allogenic mesenchymal stem cell intravenous infusion in active ankylosing spondylitis patients who failed NSAIDs: a 20-week clinical trial. Cell Transplant 2014; 23:1293-303.
21. Ryu D J, Jeon Y S, Park J S, Bae G C, Kim J S, Kim M K. Comparison of Bone Marrow Aspirate Concentrate and Allogenic Human Umbilical Cord Blood Derived Mesenchymal Stem Cell Implantation on Chondral Defect of Knee: Assessment of Clinical and Magnetic Resonance Imaging Outcomes at 2-Year Follow-Up. Cell Transplant 2020; 29: 963689720943581.
22. Alvaro-Afonso F J, Sanz-Corbalan I, Lazaro-Martinez J L, Kakagia D, Papanas N. Adipose-Derived Mesenchymal Stem Cells in the Treatment of Diabetic Foot Ulcers: A Review of Preclinical and Clinical Studies. Angiology 2020; 71:853-63.
23. Le Thi Bich P, Nguyen Thi H, Dang Ngo Chau H, Phan Van T, Do Q, Dong Khac H, et al. Allogeneic umbilical cord-derived mesenchymal stem cell transplantation for treating chronic obstructive pulmonary disease: a pilot clinical study. Stem Cell Res Ther 2020; 11:60.
24. Davies L C, Heldring N, Kadri N, Le Blanc K. Mesenchymal Stromal Cell Secretion of Programmed Death-1 Ligands Regulates T Cell Mediated Immunosuppression. Stem Cells 2017; 35: 766-76.
25. Guan Q, Li Y, Shpiruk T, Bhagwat S, Wall D A. Inducible indoleamine 2,3-dioxygenase 1 and programmed death ligand 1 expression as the potency marker for mesenchymal stromal cells. Cytotherapy 2018; 20:639-49.
26. Gu Y Z, Xue Q, Chen Y J, Yu G H, Qing M D, Shen Y, et al. Different roles of PD-L1 and FasL in immunomodulation mediated by human placenta-derived mesenchymal stem cells. Hum Immunol 2013; 74:267-76.
27. Kim J M, Chen D S. Immune escape to PD-L1/PD-1 blockade: seven steps to success (or failure). Ann Oncol 2016; 27:1492-504.
28. Guerrouahen B S, Sidahmed H, Al Sulaiti A, Al Khulaifi M, Cugno C. Enhancing Mesenchymal Stromal Cell Immunomodulation for Treating Conditions Influenced by the Immune System. Stem Cells Int 2019; 2019:7219297.
29. Fan X L, Zhang Y, Li X, Fu Q L. Mechanisms underlying the protective effects of mesenchymal stem cell-based therapy. Cell Mol Life Sci 2020; 77:2771-94.
30. Van Pham P, Truong N C, Le P T, Tran T D, Vu N B, Bui K H, et al. Isolation and proliferation of umbilical cord tissue derived mesenchymal stem cells for clinical applications. Cell Tissue Bank 2016; 17:289-302.
31. Pham L H, Vu N B, Van Pham P. The subpopulation of CD105 negative mesenchymal stem cells show strong immunomodulation capacity compared to CD105 positive mesenchymal stem cells. Biomed Res Ther 2019; 6:3131-40.
32. Dominici M, Le Blanc K, Mueller I, Slaper-Cortenbach I, Marini F, Krause D, et al. Minimal criteria for defining multipotent mesenchymal stromal cells. The International Society for Cellular Therapy position statement. Cytotherapy 2006; 8:315-7.
33. Nakamura A, Dohi Y, Akahane M, Ohgushi H, Nakajima H, Funaoka H, et al. Osteocalcin secretion as an early marker of in vitro osteogenic differentiation of rat mesenchymal stem cells. Tissue Eng Part C Methods 2009; 15:169-80.
34. Trentz O A, Arikketh D, Sentilnathan V, Hemmi S, Handschin A E, de Rosario B, et al. Surface proteins and osteoblast markers: characterization of human adipose tissue-derived osteogenic cells. Eur J Trauma Emerg Surg 2010; 36:457-63.
35. Sun H, Ye F, Wang J, Shi Y, Tu Z, Bao J, et al. The upregulation of osteoblast marker genes in mesenchymal stem cells prove the osteoinductivity of hydroxyapatite/tricalcium phosphate biomaterial. Transplant Proc 2008; 40:2645-8.
36. Miron R J, Zhang Y F. Osteoinduction: a review of old concepts with new standards. J Dent Res 2012; 91:736-44.
37. Sive J I, Baird P, Jeziorsk M, Watkins A, Hoyland J A, Freemont A J. Expression of chondrocyte markers by cells of normal and degenerate intervertebral discs. Mol Pathol 2002; 55:91-7.
38. Stoop R, Albrecht D, Gaissmaier C, Fritz J, Felka T, Rudert M, et al. Comparison of marker gene expression in chondrocytes from patients receiving autologous chondrocyte transplantation versus osteoarthritis patients. Arthritis Res Ther 2007; 9:R60.
39. Clouet J, Grimandi G, Pot-Vaucel M, Masson M, Fellah H B, Guigand L, et al. Identification of phenotypic discriminating markers for inter29. Fan X L, Zhang Y, Li X, Fu Q L. Mechanisms underlying the protective effects of mesenchymal stem cell-based therapy. Cell Mol Life Sci 2020; 77:2771-94.
40. D'Oliveira A Jr, Machado P, Bacellar O, Cheng L H, Almeida R P, Carvalho E M. Evaluation of IFN-gamma and TNF-alpha as immunological markers of clinical outcome in cutaneous leishmaniasis. Rev Soc Bras Med Trop 2002; 35:7-10.
41. Cavalcanti Y V, Brelaz M C, Neves J K, Ferraz J C, Pereira V R. Role of TNF-Alpha, IFN-Gamma, and IL-10 in the Development of Pulmonary Tuberculosis. Pulm Med 2012; 2012:745483.
42. Mehta N N, Teague H L, Swindell W R, Baumer Y, Ward N L, Xing X, et al. IFN-γ and TNF-α synergism may provide a link between psoriasis and inflammatory atherogenesis. Sci Rep 2017; 7:1383

What is claimed is:

1. A method of manufacturing a stem cell sheets, comprising:
(a) obtaining mesenchymal stem cells;
(b) proliferating mesenchymal stem cells that contain programmed death ligands one (PD-L1) to obtain programmed death ligand one mesenchymal stem cells (PD-L1 MSCs);
(c) sorting said PD-L1 MSCs to select PD-L1 positive MSCs (PD-L1⁺ MSCs);
(d) forming PD-L1⁺ MSCs sheets by mixing said PD-L1⁺ MSCs with platelet rich plasma solution and CaCl₂); and
(e) differentiating said PD-L1⁺ MSCs sheets into osteoblasts sheets and chondroblasts using a first medium and a second medium respectively in a predetermined activation condition.

2. The method of claim 1 wherein said step (a) of obtaining mesenchymal stem cells further comprising isolating said mesenchymal stem cells from from human umbilical cord tissues.

3. The method of claim 2 wherein said step (a) further comprises washing said human umbilical cord tissues twice with a washing buffer of antibiotic mycotic.

4. The method of claim 2 wherein said step (a) further comprises dissecting said human umbilical cord tissues to fragments of 1-2 mm².

5. The method of claim 4 wherein said step (b) of proliferating mesenchymal stem cells that contain programmed death ligands one (PD-L1) further comprises: placing said fragments in a T-75 cm² flask with a mesenchymal stem cell culture medium for five days; replenishing said mesenchymal stem cell culture medium every five days until said mesenchymal stem cells cover at least 70% surface area of said T-75 cm² flask.

6. The method of claim 5 wherein said step (b) of of proliferating mesenchymal stem cells that contain programmed death ligands one (PD-L1) further comprises: removing said mesenchymal stem cells using a detachment reagent; and repeating said steps of replenishing and removing until said mesenchymal stem cells for five more times.

7. The method of claim 6 wherein said step (b) of of proliferating mesenchymal stem cells that contain programmed death ligands one (PD-L1 MSCs) further comprises: washing said PD-L1 MSCs with a phosphate buffered saline.

8. The method of claim wherein said step (c) of selecting only PD-L1 positive (PD-L1⁺ MSCs) from said PD-L1 further comprises staining said PD-L1 MScs anti programmed death ligand one antibody conjugated with Phycoerythrin (PE).

9. The method of claim 8 wherein said step (c) of selecting only PD-L1 positive (PD-L1⁺ MSCs) from said PD-L1 further comprises performing flow cytometry using a fluorescent activated cell sorting (FACS) tests on said PD-L1 MSCs against a plurality of cluster of differentiations (CD) and human leukocytes antigens (HLA).

10. The method of claim 9 wherein said plurality of CDs further comprises: CD14, CD34, CD44, CD45, CD73, CD90, and CD105.

11. The method of claim 10 wherein said step (c) of selecting only PD-L1 positive (PD-L1⁺ MSCs) from said PD-L1 further comprises selecting said PD-L1 MSCs that are positive to said CD44, CD73, CD90, and CD105 from said FACS tests.

12. The method of claim 11 wherein said step (c) of selecting only PD-L1 positive (PD-L1⁺ MSCs) from said PD-L1 further comprises selecting said PD-L1 MSCs that are negative to said CD14, CD34, CD45, and said HLA-DR from said FACS tests to obtain said PD-L1⁺ MSCs.

13. The method of claim 12 wherein said step (c) of selecting PD-L1 positive (PD-L1+ MSCs) from said PD-L1 further comprises maintaining said PD-L1⁺ MSCs in a buffer with a culture medium supplemented with a 2× antibiotic mycotic substance at 37° C.

14. The method of claim 12 wherein said step (c) of selecting PD-L1 positive (PD-L1+ MSCs) from said PD-L1 further comprises transferring said PD-L1⁺ MSCs into a container.

15. The method of claim 14 wherein said step (c) of selecting PD-L1 positive (PD-L1+ MSCs) from said PD-L1 further comprises replenishing said culture medium supplemented with said 2× antibiotic mycotic substance inside said container every 24 hours.

16. The method of claim 15 wherein said step (c) of selecting PD-L1 positive (PD-L1+ MSCs) from said PD-L1 further comprises repeating said step replenishing unit said PD-L1⁺ MSCs cover at least 70% of a surface area of said container.

17. The method of claim 16 wherein said step (d) of forming PD-L1⁺ MSCs sheets by mixing said PD-L1⁺ MSCs with platelet rich plasma solution and CaCl₂) further comprises said CaCl₂) comprises 5 μl to 20 μl of CaCl₂) solution with a concentration is from 0.3 M to 1.8 M per ml of said platelet rich plasma solution.

18. The method of claim 17 wherein each of said PD-L1⁺ MSCs sheets have a thickness of 100 μm to 3 mm.

19. The method of claim 18 wherein in said step (e) said first medium is an osteoblast differentiation medium and said second medium is a chondroblast differentiation medium.

20. The method of claim 19 wherein said step (e) further comprises adding 5% to 20% insulin and growth factors.
